(19) United States
(12) Patent Application Publication
(10) Pub. No.: US 2024/0164630 A1
(43) Pub. Date: May 23, 2024

(54) INTRAORAL SCANNER

(71) Applicant: Luís António Dos SANTOS REDINHA, Paco de Arcos (PT)

(72) Inventor: Luís António Dos SANTOS REDINHA, Paco de Arcos (PT)

(21) Appl. No.: 18/281,792

(22) PCT Filed: Sep. 7, 2021
(86) PCT No.: PCT/IB2021/058124

§ 371 Date: Sep. 13, 2023

(30) Foreign Application Priority Data
Mar. 26, 2021 (PT) 117148

(51) Int. Cl. A61B 1/24; A61B 1/00; A61B 90/00; A61C 9/00

(52) U.S. Cl. CPC A61B 1/24; A61B 1/0005; A61B 1/00052; A61B 1/00105; A61C 9/0053; A61B 2090/068

(57) ABSTRACT

The present invention relates to an intraoral scanner for acquisition of the natural position of the head, three-dimensional orientation of the occlusal plane position and facial aesthetic references, and in order for it to achieve this objective the intraoral scanner (3) incorporates a device that allows the acquisition of information, said device being a level, which may be double bubble level (4), of the patient's natural position of the head as well as the three-dimensional spatial orientation of the occlusal plane and its transfer to the digital articulators, with the respective data acquisition protocol.

### TECHNICAL FIELD OF THE INVENTION

The present invention falls within the field of medical devices, specifically medical devices for use in dentistry, in particular intraoral scanning devices, even more particularly intraoral scanners for acquisition of the natural head position, three-dimensional orientation of the occlusal plane position and facial aesthetic references.

### TECHNICAL SCOPE OF THE INVENTION

The use of Intra Oral Scanners (TOS) is a growing and irreversible trend in the various fields of dental medicine. There is already a full digital flow in the areas of oral rehabilitation and orthodontics, and this will gradually lead to the disappearance of analogue workflows. However, the information collected by the current Intraoral Scanners is not sufficient for complex cases of oral rehabilitation and clear aligner systems. In these cases, the additional information provided by the current analogue systems, such as the use of the Face Arch, and other analogue systems such as Plane System by Udo Plaster, Kois DentoFacial Analyser, Ditramax, Aesthetic Cross Reference, etc. . . .

The data collected by these complementary analogue systems may relate to functional elements, which focus on the functional relation existing between the spatial position of the jaws and the Temporomandibular Joint (TMJ) or elements such as:
three-dimensional (3D) position of the upper jaw and orientation of the occlusal plane in relation to the face;
interpupillary line and natural head position; Some systems have attempted to integrate in the same protocol the two components although with only relative success, as explained below.

The subsequent digitalisation of the data obtained by these systems currently allows the achievement of digital workflows.

However, for the effective creation of a fully digital workflow, complementary tools/protocols similar to the analogue ones previously mentioned are needed, since the digital prints are currently transferred to the screen in a horizontal position that does not represent the 3D spatial position present in the patient and the positioning when mounting in the digital articulator is done based upon mean values or performed manually by the operator, trying to reproduce the 3D spatial position of the maxilla present in the patient through visual information and, for instance, comparatively, an example of this is the use of photographs.

### STATE OF THE ART OF THE INVENTION

Although there are currently some systems available on the market that attempt to provide this information digitally, such as the face scanner products Sicat by Sirona and JMA System by Zebris, their cost, complexity and time-consuming use make them expensive and inappropriate for dentists.

The incorporation of a virtual face bow (VFB) in the scanning protocol and assembly in virtual articulator for ICS has already been proposed, by the inventor of this patent application, in publication WO2016/055932A1, which discloses a correct acquisition of the functional components. However, due to its characteristics, the incorporation of a virtual face bow does not allow, in some cases, the correct determination of vital elements for the treatment planning, since it has limitations in determining the natural head position (NHP) and also in the three-dimensional spatial orientation of the maxilla regarding prosthodontic and orthodontic aesthetic determinants.

### SUMMARY OF THE INVENTION

The present invention solves the above mentioned problems since it relates to an intraoral scanner for the acquisition of the natural position of the head, three-dimensional orientation of the occlusal plane position and facial aesthetic references. In order to achieve this objective, the intraoral scanner incorporates a device which allows the acquisition of information of the patient's natural head position, as well as the 3D spatial orientation of the occlusal plane and its transfer to the digital articulators, and it is associated with the respective data acquisition protocol.

### DESCRIPTION OF THE FIGURES

FIG. 1—Representation of the present invention, with orientation of the information collection system (2) of the intraoral scanner (3), in relation to the object to be scanned in a position exactly orthogonal to the vertical plane defined by a plumb line (1), by checking the positioning of the level (4), in this representation a double bubble level.

FIG. 2—Representation of the present invention in a preferred embodiment in which the bubble level is an analogue double bubble level (4).

FIG. 3—Representation of the present invention in a preferred embodiment, in which the bubble level is an analogue circular bubble level (5).

FIG. 4—Representation of the intraoral scanner (3), with a double bubble level (4).

FIGS. 5 and 6—Representations of the present invention in a preferred embodiment, in which the level is digital (6), comprising an electronic display (7) on the body of the intraoral scanner, in various positions provided that, the allow its reading.

FIG. 7—Representation of the new acquisition method and its steps A, B and C, described herein.

FIG. 8—Representation of step D of the new method of transfer to the Virtual Articulator,

FIG. 9—Representation of step B of the new method of transfer to the Virtual Articulator.

FIG. 10—Representation of step F of the new method of transfer to the Virtual Articulator.

FIG. 11—Representation of step G of the new method of transfer to the Virtual Articulator.

FIGS. 12, 13 and 14—Representation of steps H, I (I1, I2) and of the new method of transfer to the Virtual Articulator H.

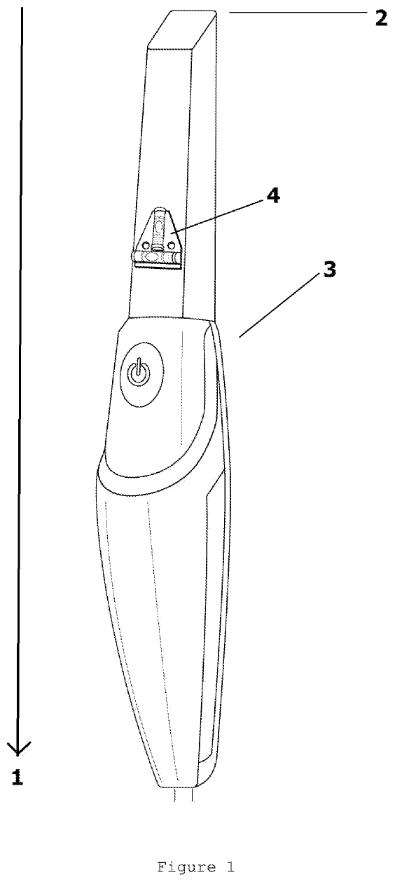

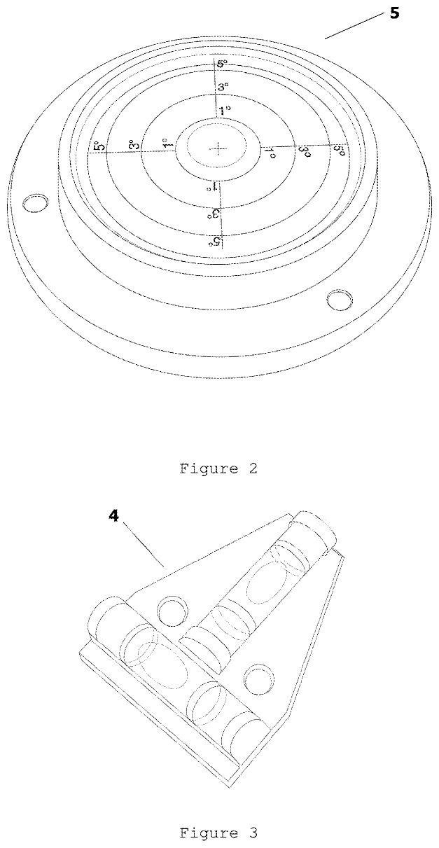

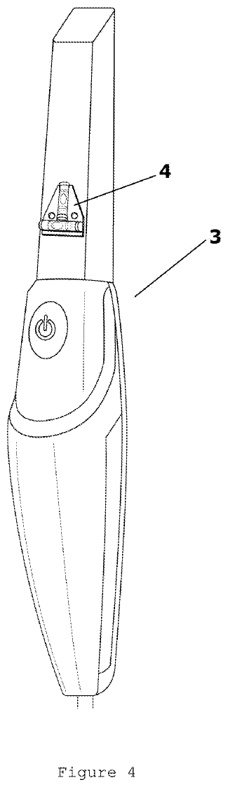

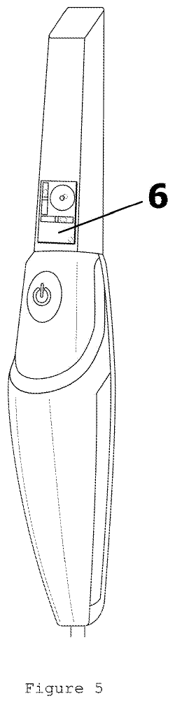

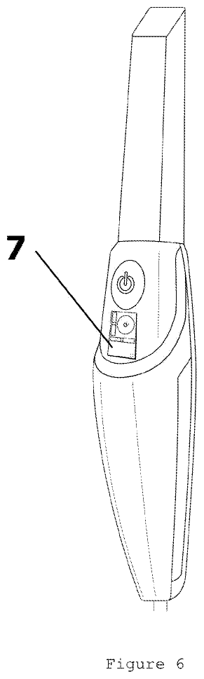

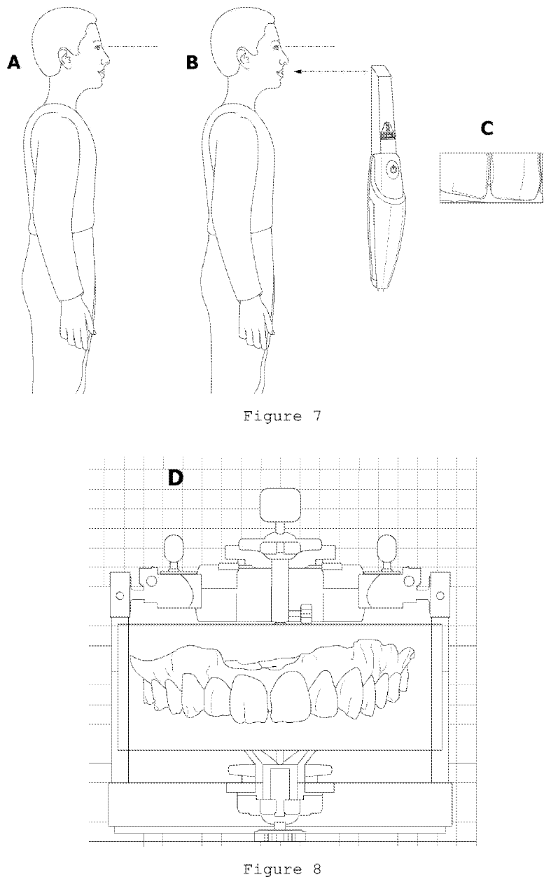

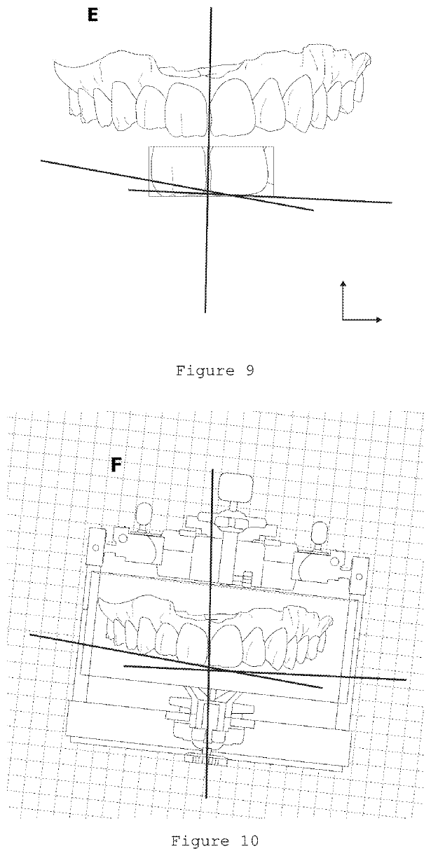

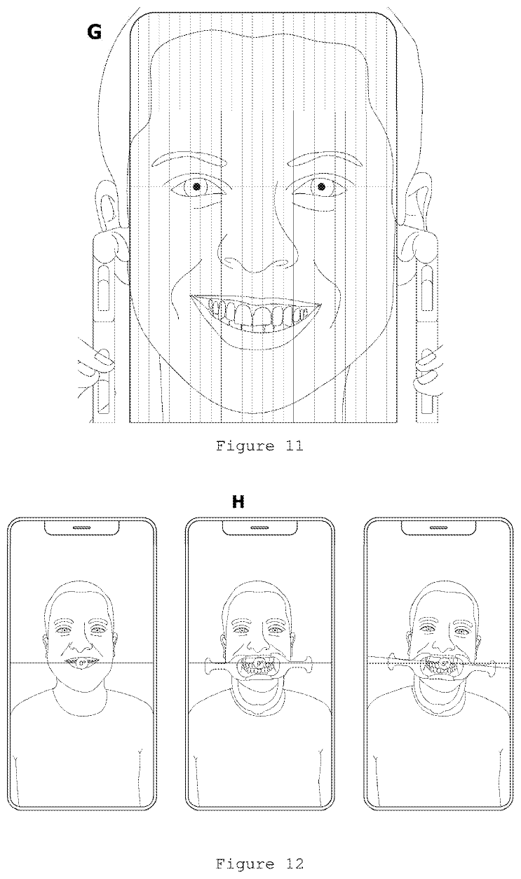

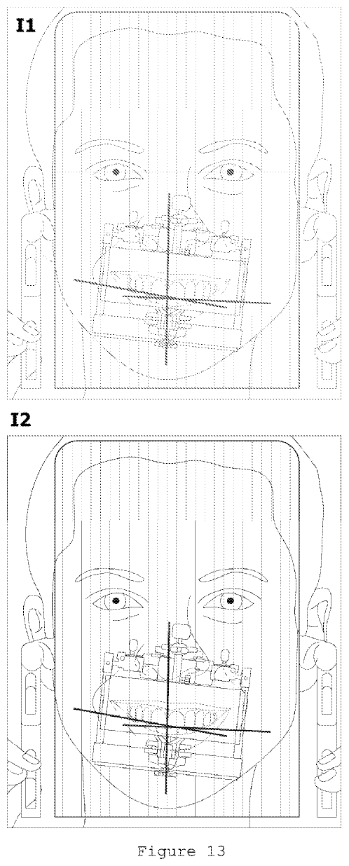

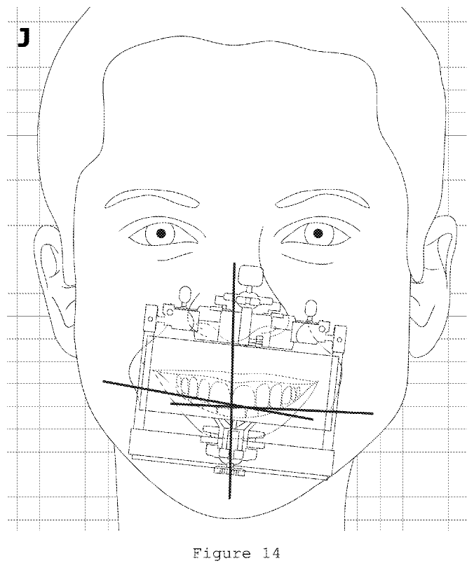

### DESCRIPTION OF THE INVENTION

The present invention relates to an intraoral scanner (3) which, in particular, allows the acquisition of the natural head position (NHP), the three-dimensional orientation of the occlusal plane position and the facial aesthetic references, comprising a bubble level, which may be analogue (4)(5) or digital (6), enabling the orientation of the information collection system (2) of the intraoral scanner (3) in relation to the object to be scanned in a position exactly orthogonal to the vertical plane defined by a plumb line (1).

The bubble level is inserted or integrated into the intraoral scanner by means of a mechanical or magnetic retention fitting.

In a preferred embodiment, the bubble level is either an analogue double bubble (4) or an analogue circular bubble (5) or a digital one (6). An analogue level (4) (5) is always guaranteed to function, a digital level (6) must be powered by a power source, e.g. batteries. However, this type of digital level (6) has higher levels of precision intended for works which require it.

In a preferred embodiment, and in case the level is digital (6), besides the fitting being possible by mechanical or magnetic retention, the fitting may also be of the camera “flash” type that allows for power supply.

In a preferred embodiment, and in case the level is digital (6), it can comprise an electronic display (7) on the body of the intraoral scanner, in various positions as long as they allow its reading.

The present invention implements a new method for acquiring the natural head position, the three-dimensional orientation of the occlusal plane position and the facial aesthetic references, which comprises the following steps:
A. after scanning the maxillary arches, with or without performing the protocol for incorporating virtual face bow, the patient stands in a relaxed position and looks straight ahead at fixed point on the horizon, keeping that position;
B. the patient lifts the upper lip in order to expose the upper central incisors or he/she can use a (spring) lip retractor for the same purpose;
C. the intraoral scanner body (3) is levelled so that the acquisition beam (2) is orthogonal to the vertical plane of the plumb line (1), then the maxillary central incisors are scanned in such a way as to acquire the interincisor midline and the maximum extension of the incisor edges allowed by the objective lens of the intraoral scanner (3);
D. mounting in the digital articulator, using mean values or through the virtual face bow protocol;
E. superimposing the image of the upper incisors obtained in the patient's natural head position onto the upper model mounted on the virtual articulator;
F. three-dimensional orientation of the articulator;
G. the operator works from a real “line of sight” perspective and maintains the functional relation of the jaws with the condyles of the temporomandibular joint (TMJ);
H. acquisition of extra-oral photograph of the patient, which can be done using a mobile device with an application like “Spirit Level Camera+”, that through the inclinometer level allows the incorporation of the natural position of the head, as well as the collection of information about the facial aesthetic determinants;
I. superimposition of extra oral photographs taken with the patient in the natural head position and which can be executed with the use of a camera with inclinometer adjusted to the vertical of the plumb line (1); or
J. superimposing a face scanner.

This new method of Facial Aesthetic References Acquisition allows the placement of the facial aesthetic determinants in an exact position in relation to the occlusal plane of the patient, thus providing a facial analysis with a high level of accuracy.

As will be evident to a person skilled in the art, the present invention should not be limited to the embodiments described herein, with a number of changes being possible, which remain within the scope of this invention.

Of course, the preferred embodiments above described are combinable in the different possible forms, the repetition of all such combinations being herein avoided.

1. Intraoral scanner (3) comprising an information collection system (2) characterized in that it comprises a bubble level (4)(5)(6) by means of a mechanical or magnetic retention fitting, allowing orientation of the information collection system (2) of the intraoral scanner (3) with respect to an object to be scanned in a position orthogonal to the vertical plane defined by a plumb line (1.

2. Intraoral scanner (3) according to claim 1, characterized in that the bubble level (4) is an analogue double bubble level.

3. Intraoral scanner (3) according to claim 1, characterized in that the bubble level (5) is an analogue circular bubble level.

4. Intraoral scanner (3) according to claim 1, characterized in that the bubble level (6) is a digital one.

5. Intraoral scanner (3) according to claim 1, characterized in that the fitting is of mechanical or magnetic retention, or of the camera “flash” type.

6. Intraoral scanner (3) according to claim 1, characterized in that the level (6) comprises an electronic display (7) in the body of the intraoral scanner (3).

7. A method for acquisition of the natural head position, three-dimensional orientation of the occlusal plane position and facial aesthetic references comprising the following steps:
A. after scanning the maxillary arches, with or without performing the protocol for incorporating a virtual face bow, the patient stands in a relaxed position and looks straight ahead at a fixed point on the horizon, keeping that position;
B. the patient lifts the upper lip in order to expose the upper central incisors or he/she can use a (spring) lip retractor for the same purpose;
characterized in that
C. the intraoral scanner body (3) according to claim 1, is levelled so that the acquisition beam (2) is orthogonal to the vertical of the plumb line (1), then the maxillary central incisors are scanned in such a way as to acquire the interincisor midline and the maximum extension of the incisor edges allowed by the objective lens of the intraoral scanner (3);
D. mounting in the digital articulator, using mean values or through the virtual face bow protocol;
E. superimposing the image of the upper incisors obtained in the patient's natural head position onto the upper model mounted on the virtual articulator;
F. three-dimensional orientation of the said articulator;
G. the operator works from a real “line of sight” perspective, maintaining the functional relation of the jaws with the condyles of the temporomandibular joint (TMJ);
H. acquisition of extra-oral photograph of the patient and incorporation of the natural position of the head, as well as the collection of information about the facial aesthetic determinants;
I. superimposition of extra oral photographs taken with the patient in the natural head position; or
J. superimposing a face scanner.

8. Method according to claim 7, characterized in that the photographs are executed using a:
a. camera with an inclinometer set to the vertical of the plumb line (1); and/or
b. mobile device with a “Spirit Level Camera+” type application.
